(19) United States
(12) Patent Application Publication
(10) Pub. No.: US 2026/0355426 A1
(43) Pub. Date: Nov. 12, 2026

(54) A SUB-ASSEMBLY OF A MEDICAMENT DELIVERY DEVICE

(71) Applicant: SHL Medical AG, Zug (CH)

(72) Inventors: Johan Zander, Nacka Strand (SE); Munib Baber, Stockholm (SE); Hsuan Wang, Zug (CH); Meng-Jhen Chiou, Taoyuan (TW)

(21) Appl. No.: 19/123,863

(22) PCT Filed: Oct. 20, 2023
(86) PCT No.: PCT/EP2023/079219

§ 371 Date: Apr. 24, 2025

(30) Foreign Application Priority Data
Nov. 4, 2022 (EP) 22205555.0

(51) Int. Cl. A61M 5/34; A61M 5/20; A61M 5/24; A61M 5/315

(52) U.S. Cl. CPC A61M 5/347; A61M 5/2033; A61M 5/31578; A61M 2005/2013; A61M 2005/2474

(57) ABSTRACT

The present disclosure provides a sub-assembly of a medicament delivery device, the sub-assembly including a tubular housing extending along a longitudinal axis between a proximal end and a distal end. The sub-assembly further includes a delivery member guard telescopically arranged within the tubular housing. The delivery member guard comprises a distal portion and a proximal portion configured to surround a medicament delivery member of the medicament delivery device. The distal portion of the delivery member guard comprises a support surface directed towards the longitudinal axis. The sub-assembly further includes a plunger rod configured to be connected to a power source that is configured to move the plunger rod towards the proximal end of the housing. The plunger rod comprises a proximally directed surface. The sub-assembly further includes a retainer axially immovably arranged within the housing. The retainer is configured to releasably hold the plunger rod immovably relative to the housing and is configured to be released by the distal movement of the delivery member guard relative to the housing.

### TECHNICAL FIELD

The present disclosure generally relates to a sub-assembly of a medicament delivery device, and particularly to a sub-assembly of a medicament delivery device comprising a delivery member guard with a support surface and a retainer.

### BACKGROUND

Medicament delivery devices such as pen-type manual injectors or auto-injectors are generally known for the self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users are without professional training or are in an emergency situation, the users can easily and properly use the medicament delivery devices.

Even though many of the devices on the market, as well as the ones described above, have their respective advantages, there is still room for improvement.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device, the sub-assembly comprising: a tubular housing extending along a longitudinal axis between a proximal end and a distal end; a delivery member guard telescopically arranged within the tubular housing. The delivery member guard comprises a distal portion and a proximal portion configured to surround a medicament delivery member of the medicament delivery device; wherein the distal portion of the delivery member guard comprises a support surface directed towards the longitudinal axis. A plunger rod is configured to be connected to a power source that is configured to move the plunger rod towards the proximal end of the housing. The plunger rod comprises a proximally directed surface. A retainer is axially immovably arranged within the housing. The retainer comprises a flexible wall arranged between the plunger rod and the distal portion of the delivery member guard in the direction transverse to the longitudinal axis. The flexible wall comprises a distally directed surface. The flexible wall is radially movable relative to the plunger rod between a first position where the distally directed surface of the flexible wall is engaged with the proximally directed surface of the plunger rod and a second position where the distally directed surface of the flexible wall is disengaged from the proximally directed surface of the plunger rod. The delivery member guard is movable relative to the retainer between a distal position where the support surface of the distal portion of the delivery member guard is adjacent to the flexible wall of the retainer so that the flexible wall is retained in the first position and a proximal position where the support surface is spaced apart from the flexible wall.

Preferably, according to another embodiment, the retainer is attached to the housing.

Preferably, according to another embodiment, the housing comprises a distally directed surface. The retainer comprises a proximally directed surface adjacent to the distally directed surface of the housing such that the retainer is axially immovable relative to the housing in the proximal direction of the housing.

Preferably, according to another embodiment, the retainer is rotatable around the longitudinal axis relative to the housing.

Preferably, according to another embodiment, the retainer comprises a ledge extending from a wall of the retainer towards the longitudinal axis. The ledge is proximally positioned relative to the distally directed surface of the flexible wall of the retainer. The ledge is aligned with the proximally directed surface of the plunger rod.

Preferably, according to another embodiment, the retainer comprises a retainer body.

Preferably, according to another embodiment, the flexible wall of the retainer is a flexible arm.

Preferably, according to another embodiment, the flexible arm is defined by two elongated cut-outs extending in the direction of the longitudinal axis in a wall of the retainer body.

Preferably, according to another embodiment, the retainer comprises a support ledge extending from the flexible wall towards the longitudinal axis. The distally directed surface of the flexible wall is the support ledge.

Preferably, according to another embodiment, the distal portion of the delivery member guard is attached to the proximal portion via a form-fit or press-fit connection.

Preferably, according to another embodiment, the plunger rod comprises an elongated body extending in the direction of the longitudinal axis.

Preferably, according to another embodiment, the plunger rod comprises a retaining ledge extending from the elongated body away from the longitudinal axis. The retaining ledge comprises the proximally directed surface.

Preferably, according to another embodiment, a recess/cut-out is in a wall of the elongated body of the plunger rod. The recess/cut-out is open to the direction transverse to the longitudinal axis. The proximally directed surface is defined by an edge of the recess/cut-out.

Preferably, according to another embodiment, the sub-assembly comprises a biasing member configured to bias the delivery member guard in the proximal direction relative to the housing from the distal position to the proximal position.

Preferably, according to another embodiment, the delivery member guard is configured to be manually moved from the distal position to the proximal position.

Preferably, according to another embodiment, one of the distally directed surfaces of the retainer and the proximally directed surface of the plunger rod is bevelled relative to the longitudinal axis.

Preferably, according to another embodiment, the sub-assembly is used in a medicament delivery device comprising a power source at least partially arranged within the housing. The power source is connected to the plunger rod. The power source is configured to move the plunger rod in the proximal direction relative to the housing. A medicament container comprises a container body and a distal seal axially movable relative to the container body. The medicament container is proximally positioned relative to the plunger rod within the housing. When the delivery member guard is in the proximal position, the plunger rod is configured to be moved by the power source to urge the distal seal of the medicament container in the proximal direction relative to the container body.

Preferably, according to another embodiment, the power source is a spring, a gas canister, or an electric motor.

Preferably, according to another embodiment, the spring is a compression spring, a torsion spring, a band spring, or a tension spring.

Preferably, according to another embodiment, the medicament delivery device comprises a delivery member assembly.

Preferably, according to another embodiment, the medicament container comprises a pierceable proximal seal.

Preferably, according to another embodiment, the delivery member assembly comprises a delivery member hub, a delivery member attached to the delivery member hub, and an attaching member attached to the proximal end of the housing.

Preferably, according to another embodiment, the delivery member hub is connected to the attaching member via a bayonet connection or a helical connection so that the delivery member hub can be manually moved towards the pierceable proximal seal relative to the housing.

Preferably, according to another embodiment, the sub-assembly is used in a medicament delivery device comprising a multiple-chamber medicament container arranged within the carrier.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery member is an injection needle or a spray nozzle.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example, pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a sub-assembly of the invention in an initial state;

FIG. 2 schematically shows a perspective view of the sub-assembly of FIG. 1 in a ready-to-be-used state;

FIG. 3 schematically shows a perspective view of components of the sub-assembly of FIG. 1;

FIG. 4 schematically shows a cross-section view of one embodiment of the sub-assembly of FIG. 1;

FIG. 5 schematically shows a perspective view of a rotator of another embodiment of the sub-assembly of FIG. 1.

FIGS. 6-7 schematically show cress-section views of another embodiment of the sub-assembly of FIG. 1;

FIG. 8 schematically shows a perspective view of a proximal end of the sub-assembly of FIG. 1;

FIG. 9 schematically shows a cross-section view of a medicament delivery device comprising the sub-assembly of FIG. 1;

FIG. 10 schematically shows a perspective view of the sub-assembly of FIG. 1 comprising a cap.

FIG. 11 schematically shows a perspective view of the sub-assembly of FIG. 1 comprising a cap and a ring in another embodiment.

FIGS. 12A-12B schematically show perspective views of the sub-assembly of FIG. 1 comprising a cap and a ring in another embodiment.

### DETAILED DESCRIPTION

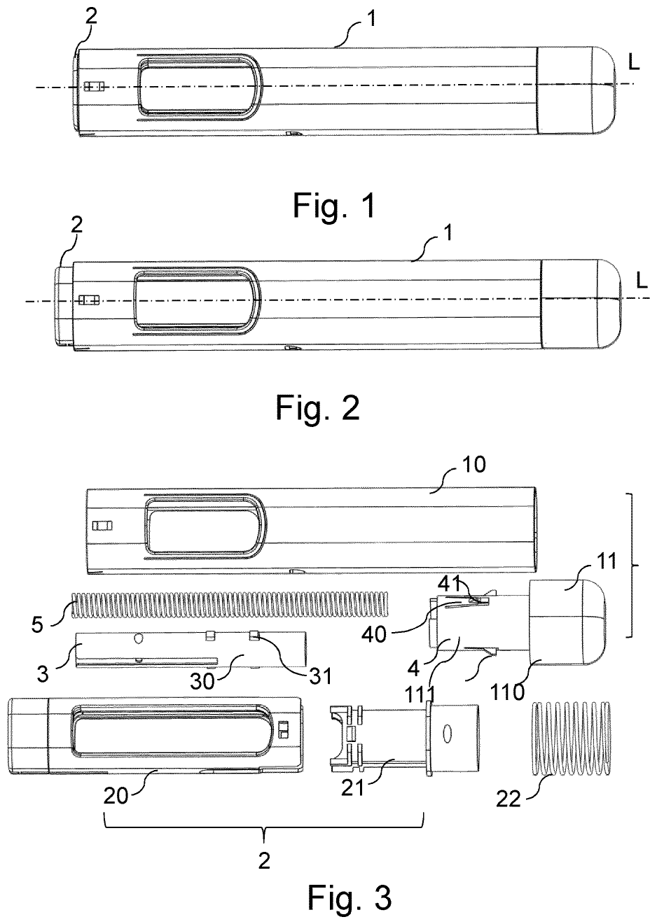

FIGS. 1-11 illustrate some examples of sub-assemblies of a medicament delivery device. The sub-assembly comprises a tubular housing 1, a delivery member guard 2, a plunger rod 3, and a retainer 4.

The tubular housing 1 extends along a longitudinal axis L between a proximal end and a distal end. The tubular housing 1 is configured to receive a medicament container of the medicament delivery device. In one example, the tubular housing 1 comprises a main body 10, and an inner housing 11, as shown in FIG. 3. In one example, the main body 10 is tubular. The main body 10 extends along the longitudinal axis L between a proximal opening and a distal opening. Preferably, the main body 10 comprises an observation window where a user of the medicament delivery device can check the medicament within the medicament container before use. The window can be a transparent portion of the main body 10, or an aperture in a wall of the main body 10. Alternatively, the main body 10 can be made of transparent material so that the window is no longer necessary for the user to observe the medicament. In a preferred example, as shown in FIG. 3, the main body 10 has a rectangular cross-section perpendicular to the longitudinal axis L, so that the medicament delivery device can be prevented from rolling on a flat surface. It should be noted that such functionality can be obtained by forming the main body 10 in any shape that has a non-circular cross-section perpendicular to the longitudinal axis L.

The inner housing 11 comprises a distal cover 110 and an extension 111. The inner housing 11 is configured to be inserted into the main body 10. In a preferred example, the extension 111 is configured to be positioned within the main body 10, and the distal cover 110 is configured to cover the distal opening of the main body 10. Alternatively, instead of being formed by multiple components, the tubular housing can be formed by a single component, depending on the design.

The delivery member guard 2 is telescopically arranged within the tubular housing 1, as shown in FIGS. 1-2. The delivery member guard 2 comprises a distal portion 21; 21′ and a proximal portion 20. The proximal portion 20 is configured to surround a medicament delivery member of the medicament delivery device. In one example as shown in FIG. 3, the proximal portion 20 is tubular. Alternatively, the proximal portion can be formed by multiple proximally extending arms. The distal portion 21; 21′ of the delivery member guard 2 comprises a support surface 21a; 21a′ directed towards the longitudinal axis L. In one example, the distal portion 21; 21′ is attached to the proximal portion 20 via a form-fit or press-fit connection. Alternatively, the delivery member guard is made as a single-piece component, meaning that the proximal portion and the distal portion are integral to one another.

The plunger rod 3 is configured to be connected to a power source 5 that is configured to move the plunger rod 3 towards the proximal end of the housing 1. In one example as shown in FIG. 3, the power source 5 is a compression spring being compressed between a part of the plunger rod 3 and a part of the tubular housing. Alternatively, the power source can be a torsion spring, a tension spring, a band spring, a compressed gas spring or an electrical motor. In one example as shown in FIG. 3, the plunger rod 3 comprises an elongated body 30 extending along the longitudinal axis from a proximal end and a distal end. The proximal end of the elongated body 30 is configured to act on a part of the medicament container and therefore expel the medicament out of the medicament container. The plunger rod 3 comprises a proximally directed surface. In one example, the plunger rod is tubular. In this example, the power source can be at least partially arranged within the plunger rod. Alternatively, the plunger rod is a solid core stick. In this example, the power source is configured to act on the distal end of the plunger rod. Alternatively, the plunger rod comprises a flange. In this example, the power source is configured to act on the flange of the plunger rod.

The retainer 4; 4′ is axially immovably arranged within the housing 1. The retainer 4; 4′ comprises a flexible wall 40; 40′ arranged between the plunger rod 3 and the distal portion of the delivery member guard 2 in the direction transverse to the longitudinal axis L. The flexible wall 40; 40′ comprises a distally directed surface releasably engaged with the proximally directed surface of the plunger rod 3.

The flexible wall 40; 40′ is radially movable relative to the plunger rod 3 between a first position where the distally directed surface of the flexible wall 40; 40′ is engaged with the proximally directed surface of the plunger rod 3 and a second position where the distally directed surface of the flexible wall 40; 40′ is disengaged from the proximally directed surface of the plunger rod 3.

The delivery member guard 2 is movable relative to the retainer 4; 4′ between a distal position where the support surface 21a; 21a′ of the distal portion of the delivery member guard 2 is adjacent to the flexible wall 40; 40′ of the retainer 4; 4′ such that the flexible wall 40; 40′ is retained in the first position and a proximal position where the support surface 21a; 21a′ is spaced apart from the flexible wall 40; 40′.

In one example, the retainer 4; 4′ is attached to the housing 1 such that the retainer 4; 4′ is axially immovable relative to the tubular housing 1. Alternatively, the retainer is axially movable relative to the tubular housing within a certain distance. In the example where the retainer is axially movable relative to the tubular housing within a certain distance, the power source can be arranged between the retainer and the plunger rod. For example, when the power source is a compression spring, the compressions spring extends from a part of the plunger rod to a part of the retainer.

In this example, the retainer is configured to be pushed by the power source in the distal direction relative to the housing after the plunger rod is released from the retainer.

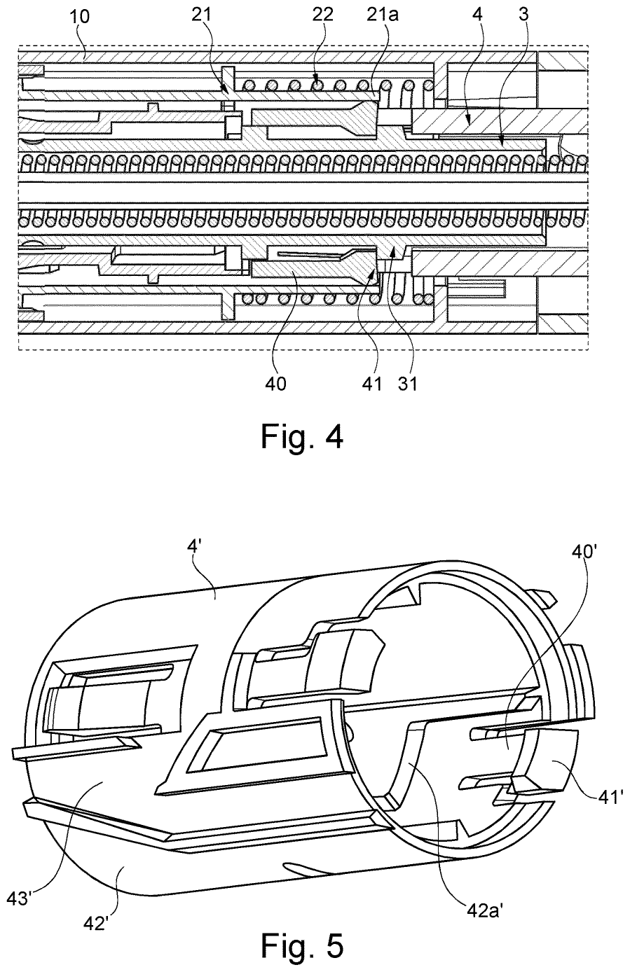

In one example where the retainer 4 is attached to the housing 1, as shown in FIGS. 3-4, the retainer 4 is a part of the tubular housing 1. In one example where the tubular housing 1 comprises the main body 10 and inner housing 11, the retainer 4 is a part of the inner housing 11. In one example, the retainer 4 is a part of the extension 111 of the inner housing 11. In this example, the retainer comprises a ledge 41 extending from a wall of the retainer 4 towards the longitudinal axis L. The retainer 4 comprises a support ledge 41 extending from the flexible wall 40 towards the longitudinal axis L. In this example, the distally directed surface of the flexible wall is the support ledge 41. The plunger rod 3 comprises a retaining ledge 31; 31′ extending from the elongated body 30 away from the longitudinal axis L. The proximally directed surface is the retaining ledge 31; 31′, as shown in FIG. 4 and FIGS. 6-7 (which will be explained later).

In a preferred example, the retainer 4 comprises a retainer body. In one example, the flexible wall 40 of the retainer 4 is a flexible arm defined by two elongated cut-outs extending in the direction of the longitudinal axis in a wall of the retainer body. In the example shown in FIG. 4, the flexible arm is directed to the distal end of the housing 1. Preferably, the support ledge 41 extends to the longitudinal axis L from a distal tip to the flexible arm 40, as shown in FIG. 4. As the support surface 21a of the delivery member guard 2 blocks the flexible arm 40, the flexible arm 40 cannot flex radially outward relative to the plunger rod 3, therefore, the plunger rod 3 is prevented from being moved relative to the retainer 4 in the proximal direction of the housing 1. When the delivery member guard 2 moves to the proximal position, the support surface 21a no longer blocks the flexible arm 40 from flexing radially outward relative to the plunger rod 3. Therefore, under the force from the power source, the support ledge 41 is pushed in the direction transverse to the longitudinal axis L, therefore, the plunger rod 3 is released from the retainer 4. In one example, one of the distally directed surface of the retainer 4; 4′ and the proximally directed surface of the plunger rod 3 is bevelled relative to the longitudinal axis L so that the support ledge 41; 41′ can be easily pushed in the direction transverse to the longitudinal axis L when the delivery member guard 2 is in the proximal position. Alternatively, the retainer comprises a resilient section defining the flexible wall. In this example, the support ledge extends from the resilient section of the retainer. When the support ledge is pushed in the direction transverse to the longitudinal axis L, the entire resilient section deforms in the direction transverse to the longitudinal axis L.

Another example is shown in FIGS. 5-7. In this example, the retainer 4′ is not a part of the housing 1. Similar as the example mentioned above, the plunger rod 3 comprises a retaining ledge 31′ extending from the elongated body 30 away from the longitudinal axis L. The proximally directed surface is the retaining ledge 31′. Furthermore, the retainer 4′ comprises a support ledge 41′ extending from the flexible wall 40′ towards the longitudinal axis L. The distally directed surface of the flexible wall 40′ is the support ledge 41′. One of the distally directed surface of the retainer 4′ and the proximally directed surface of the plunger rod 3 is bevelled relative to the longitudinal axis L so that the support ledge 41′ can be easily pushed in the direction transverse to the longitudinal axis L when the delivery member guard 2 is in the proximal position.

In this example, the retainer 4′ is attached to the housing 1. In this example, the housing 1 comprises a distally directed surface. The retainer 4′ comprises a proximally directed surface adjacent to the distally directed surface of the housing 1 such that the retainer 4′ is axially immovable relative to the housing 1 in the proximal direction of the housing 1. In a preferred example, the retainer 4′ is rotatable around the longitudinal axis L relative to the housing 1. In this example, the retainer 4′ comprises a ledge 42a′ extending from a wall 42′ of the retainer 4′ towards the longitudinal axis L. The ledge 42a′ is proximally positioned relative to the distally directed surface of the flexible wall 40′ of the retainer 4′, as shown in FIG. 5. The ledge 42a′ is aligned with the proximally directed surface of the plunger rod 3. In this example, the retainer 4′ can be used to control the movement of the plunger rod in the proximal direction relative to the housing 2 twice.

In one example, the plunger rod 3 is configured to be released twice relative to the housing. In other words, the plunger rod 3 is configured to be initially held against the force from the power source, then is configured to be released and move in the proximal direction relative to the housing 1 with a first distance under the force of the power source. The plunger rod 3 is configured to be held against the force from the power source after moving with the first distance. Afterward, the plunger rod 3 is configured to be released again and move in the proximal direction relative to the housing with the second distance under the force of the power source.

When the plunger rod moves with the first distance, a priming shot or a drug mixing operation can be performed. Furthermore, when the plunger rod moves with the second distance, an medicament delivery operation can be performed.

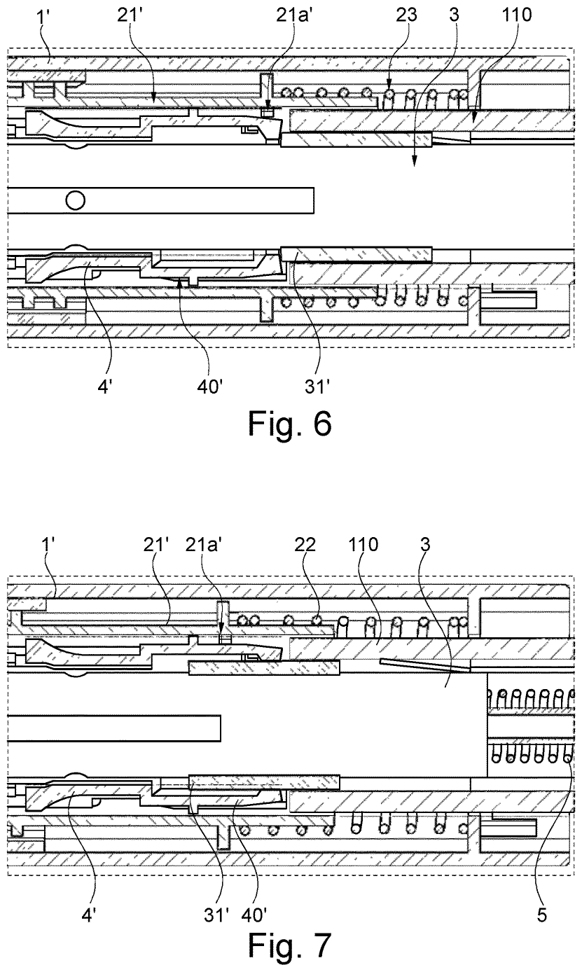

In one example where the plunger rod is configured to be released twice relative to the housing, the retainer 4′, is configured to initially hold the plunger rod 3 when the delivery member guard 2 is in the distal position. FIG. 6 shows that the delivery member guard 2 is in the distal position. When the delivery member guard 2 is in the distal position, the support surface 21a′ is lined up with the flexible wall 40′ of the retainer 4′ such that the flexible wall 40′ of the retainer 4′ is blocked from flexing radially outward relative to the retaining ledge 31′ of the plunger rod 3. FIG. 7 shows that the delivery member guard 2 is in the proximal position. When the delivery member guard 2 is in the proximal position, the support surface 21a′ no longer blocks the flexible wall 40′ of the retainer 4′ from flexing radially outward relative to the retaining ledge 31′ of the plunger rod 3; thus, under the force from the power source, the support ledge 41′ is pushed in the direction transverse to the longitudinal axis L, therefore, the plunger rod 3 is released from the retainer 4.

Once the retaining ledge 31′ of the plunger rod 3 disengages from the support ledge 41′ of the retainer 4′, the plunger rod 3 moves in the proximal direction relative to the retainer 4′. In this example, the plunger rod 3 will be held by the retainer 4′ again when the retaining ledge 31′ is engaged with the ledge 42a′ of the retainer 4′. As the retainer 4′, in this example, is rotatable relative to the tubular housing 1, the rotation of the retainer 4′ can make the ledge 42a′ of the retainer 4′ circumferentially offset to the retaining ledge 3′ of the plunger rod 3; therefore, the plunger rod 3 is no longer held by the retainer 4′, thus, the plunger rod 3 can be moved again by the power source 5 in the proximal direction relative to the tubular housing 1.

In one example, the retainer comprises a track 43′ configured to guide a movement of a rib of the delivery member guard 2. In one example, the track 43′ comprises a proximally directed surface being bevelled to the longitudinal axis L. In this example, when the delivery member guard is moved to the distal end of the housing 1, the rib of the delivery member guard can travel along the proximally directed surface of the retainer 4′ and thus rotates the retainer 4′ relative to the plunger rod 3. Therefore, the plunger rod 3 is released and can be moved in the proximal direction relative to the housing 1.

Similar to the above-mentioned example, the retainer 4′ comprises a retainer body and the flexible wall 40′ of the retainer 4′ is a flexible arm 40′ defined by two elongated cut-outs extending in the direction of the longitudinal axis L in a wall of the retainer body.

In one example, the delivery member guard 2 is configured to be moved from the distal position to the proximal position by a biasing member, e.g., a spring or a flexible arm. Alternatively, the delivery member guard 2 can be moved from the distal position to the proximal position manually by the user.

For example, the sub-assembly comprises a delivery member guard spring 22. The delivery member guard spring 22 is positioned between a part of the tubular housing 1 and a part of the delivery member guard 2. The delivery member guard spring 22 is configured to bias the delivery member guard 2 in the proximal direction relative to the tubular housing 1.

In one example, the delivery member guard 2 is tensioned in the assembling process and is maintained in the tensioned state before use. In this example, the delivery member guard is in the distal position before use. When the user plans to use the medicament delivery device, the user unlocks the delivery member guard spring 22 first; therefore, the delivery member guard spring 22 pushes the delivery member guard 2 to the proximal position. Meanwhile, the plunger rod 3 is released from the retainer 4; 4′ and is pushed in the proximal direction relative to the tubular housing 1 by the power source 5. The proximal movement of the plunger rod 3 can be used for providing a priming shot (expel contained air within the medicament container) and/or a medicament reconstitution. Therefore, the medicament can be prepared when the user plans to use the device.

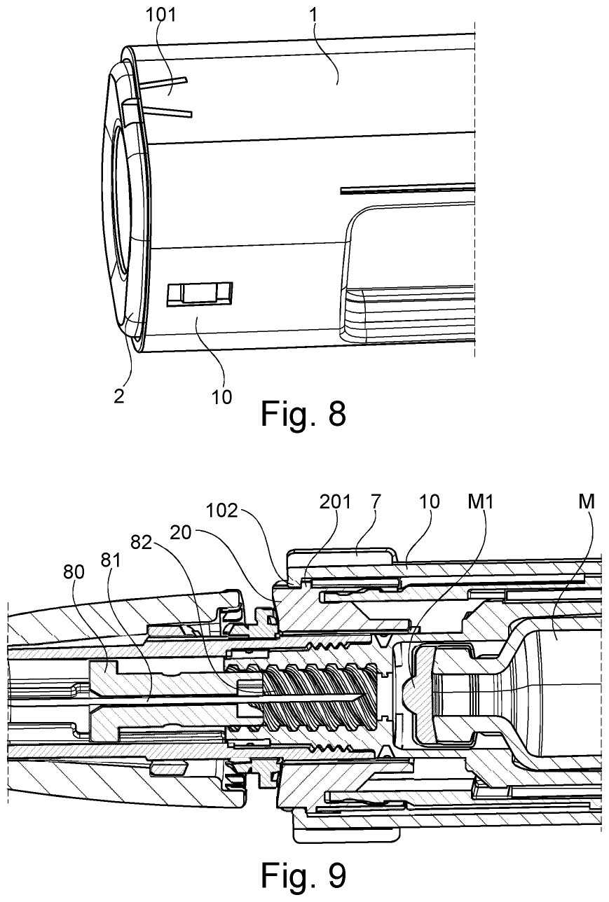

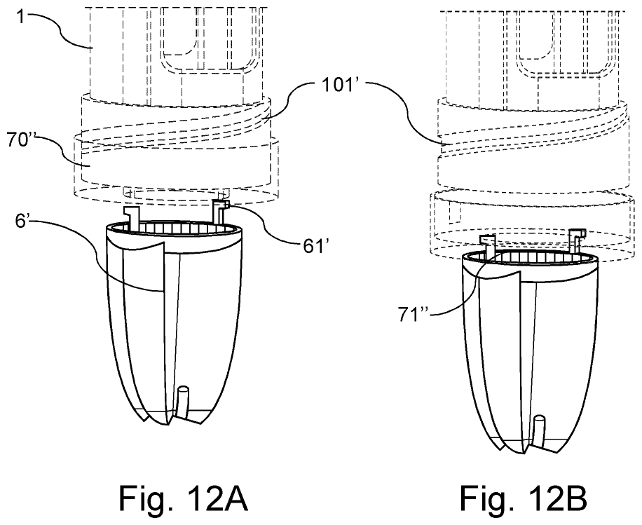

In one example, the sub-assembly comprises a locking mechanism configured to releasably lock the delivery member guard 2 from moving from the distal position to the proximal position. As shown in FIGS. 8-9, the locking mechanism comprises an arm 101 of the housing 1, a rib 201 of the delivery member guard 2 and a protecting lock 7. The arm 101 comprises the protrusion 102 extending to the longitudinal axis L. In a preferred example, the protrusion 102 is extending from a proximal tip end of the arm 101. The protrusion 102 is configured to releasably engage with a rib 201 of the delivery member guard 2. In one preferred example, the rib 201 extends from the proximal portion 20 of the delivery member guard 2, as shown in FIG. 9. The protecting lock 7 is configured to be moved by the user between a lock position where a portion of the protecting lock 7 is aligned with the arm 101 of the tubular housing 1, so that the arm 101 cannot flex radially outward and thus release the rib 201 of the delivery member guard 2 and a unlock position where the protecting lock 7 no longer blocks the arm 101 of the tubular housing 1. Once the arm 101 of the tubular housing 1 can flex radially outward, the movement of the delivery member guard 2 in the proximal direction relative to the tubular housing 1 will not be limited. In a preferred example, one of the protrusion 102 and rib 201 comprises a bevelled surface that is configured to engage with the other one of the protrusion 102 and rib 201. Alternatively, instead of the arm 101 of the tubular housing 1, the locking mechanism can be provided by a locking ring 70″ screwed to the proximal end of the tubular housing 1, as shown in FIGS. 12A-12B. As a helical screw 101′ can guide the locking ring 70″ to move in the direction of the longitudinal axis L, thus, when the locking ring 70″ is screwed on the proximal end of the tubular housing 1, the locking ring 70″ supports the delivery member guard 2 in the distal position. Once the locking ring 70″ is screwed to be removed from the tubular housing 1, the delivery member guard 2 can move to the proximal position. In a preferred example, a cap 6′ is operably connected to the locking ring 70″. For example, the cap 6′ comprises a hook 61′ configured to be snapped fit or bayonet fit to a recess 71″ arranged in a wall of the locking ring 70″. In this example, the user can removed the lock ring 70″ from the tubular housing 1 by rotating the cap 6′.

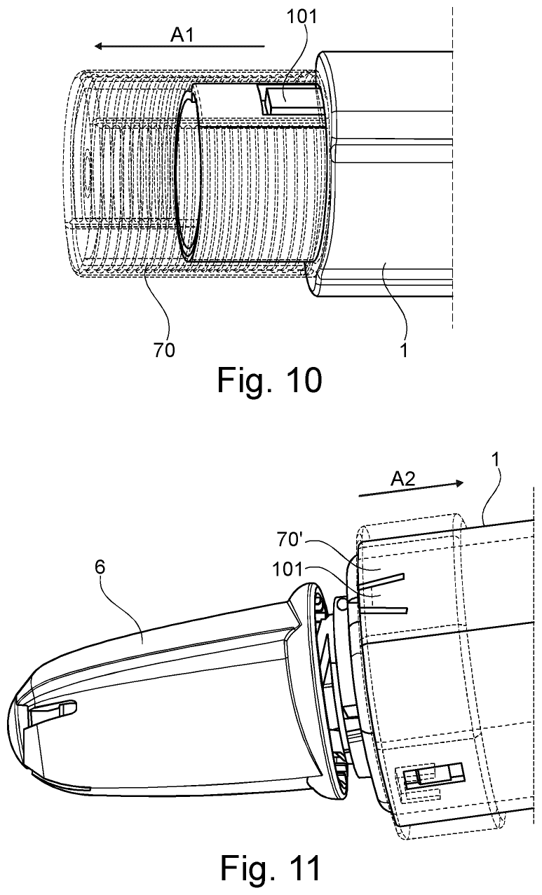

In one example, the protecting lock 7 comprises a cap 70, as shown in FIG. 10. The cap 70 is configured to be moved by the user from the housing 1 in the proximal direction A1 such that the cap 70 is removed from the tubular housing 1. The cap can be removed by pulling or turning relative to the housing 1. Alternatively, the protecting lock 7 comprises a locking ring 70′, as shown in FIG. 11. The locking ring 70′ is configured to be moved by the user in the distal direction A2 relative to the tubular housing 1. In the example, the sub-assembly optionally comprises a cap 6 that is independent to the locking ring 70′.

In one example where the delivery member guard 2 is connected to the delivery member guard spring 22, the locking mechanism is configured to lock the delivery member guard 2 against the force of the delivery member guard spring 22. Therefore, when the user moves the protecting lock 7 to the unlock position, the delivery member guard 2 can move to the proximal position under the force of the delivery member guard spring 22. For example, when the protecting lock 7 comprises the cap 70, when the user removes the cap 70, the medicament can be prepared, e.g., primed or reconstituted, at the same time. Alternatively, the delivery member guard can be pulled by the user from the distal position to the proximal position. In this example, the delivery member guard spring is optional. In this example, the user needs to move the protecting lock 7 to the unlock position first, then to pull the delivery member guard 2 in the proximal direction relative to the housing 1.

Another aspect of the invention provides a medicament delivery device comprising the sub-assembly as disclosed above. The medicament delivery device comprises a power source 5 at least partially arranged within the housing 1. The power source 5 is connected to the plunger rod 3. The power source 5 is configured to move the plunger rod 3 in the proximal direction relative to the housing 1. The medicament delivery device comprises a medicament container comprising a container body and a distal seal axially movable relative to the container body. The medicament container is proximally positioned relative to the plunger rod 3 within the housing 1. When the delivery member guard 2 is in the proximal position, the plunger rod 3 is configured to be moved by the power source 5 to urge the distal seal of the medicament container in the proximal direction relative to the container body. Therefore, the plunger rod 3 can cause the contained air within the medicament container to be expelled and/or can cause the medicament contained within different chambers of the medicament container to be mixed.

In another example, the medicament delivery device comprises a delivery member assembly. The medicament container M comprises a pierceable proximal seal M1. The delivery member assembly comprises a delivery member hub 80, a delivery member 81 attached to the delivery member hub 80, and an attaching member 82 attached to the proximal end of the housing 1. The delivery member hub 80 is connected to the attaching member 82 via a bayonet connection or a helical connection so that the delivery member hub 80 can be manually moved towards the pierceable proximal seal M1 relative to the housing 1. In one example, the delivery member hub 80 is coupled to the cap 70; 6 such that when the cap 70; 6 is moved away from the housing 1, the delivery member hub 80 is moved towards the medicament container M. In one example, the cap is configured to be removed from the housing via a rotational connection between the cap and the housing, for example, a thread connection. In this example, the thread between the cap and the housing directs to the opposite direction to the helical connection between the delivery member hub 80 and the attaching member 82. In this example, the cap and the delivery member hub 80 are coupled such that the cap is rotationally immovable and axially movable relative to the delivery member hub 80.

Furthermore, the housing of the medicament delivery device, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A sub-assembly of a medicament delivery device, the sub-assembly comprising:
a tubular housing extending along a longitudinal axis between a proximal end and a distal end;
a delivery member guard telescopically arranged within the tubular housing, wherein the delivery member guard comprises a distal portion and a proximal portion configured to surround a medicament delivery member of the medicament delivery device, wherein the distal portion of the delivery member guard comprises a support surface directed towards the longitudinal axis;
a plunger rod configured to be connected to a power source that is configured to move the plunger rod towards the proximal end of the housing, wherein the plunger rod comprises a proximally directed surface; and
a retainer axially immovably arranged within the housing, wherein the retainer comprises a flexible wall arranged between the plunger rod and the distal portion of the delivery member guard in the direction transverse to the longitudinal axis, wherein the flexible wall comprises a distally directed surface, wherein the flexible wall is radially movable relative to the plunger rod between a first position where the distally directed surface of the flexible wall is engaged with the proximally directed surface of the plunger rod and a second position where the distally directed surface of the flexible wall is disengaged from the proximally directed surface of the plunger rod,
wherein the delivery member guard is movable relative to the retainer between a distal position where the support surface of the distal portion of the delivery member guard is adjacent to the flexible wall of the retainer so that the flexible wall is retained in the first position and a proximal position wherein the support surface is spaced apart from the flexible wall.

17. The sub-assembly according to claim 16, wherein the retainer is attached to the housing.

18. The sub-assembly according to claim 16, wherein the housing comprises a distally directed surface, and wherein the retainer comprises a proximally directed surface adjacent to the distally directed surface of the housing such that the retainer is axially immovable relative to the housing in the proximal direction of the housing.

19. The sub-assembly according to claim 18, wherein the retainer is rotatable around the longitudinal axis relative to the housing.

20. The sub-assembly according to claim 19, wherein the retainer comprises a ledge extending from a wall of the retainer towards the longitudinal axis, wherein the ledge is proximally positioned relative to the distally directed surface of the flexible wall of the retainer, and wherein the ledge is aligned with the proximally directed surface of the plunger rod.

21. The sub-assembly according to claim 16, wherein the retainer comprises a retainer body, and wherein the flexible wall of the retainer is a flexible arm defined by two elongated cut-outs extending in the direction of the longitudinal axis in a wall of the retainer body.

22. The sub-assembly according to claim 16, wherein the retainer comprises a support ledge extending from the flexible wall towards the longitudinal axis, and wherein the support ledge comprises the distally directed surface.

23. The sub-assembly according to claim 16, wherein the distal portion of the delivery member guard is attached to the proximal portion via a form-fit or press-fit connection.

24. The sub-assembly according to claim 16, wherein the plunger rod comprises an elongated rod body extending in the direction of the longitudinal axis, wherein the plunger rod comprises a retaining ledge extending from the rod body away from the longitudinal axis, and wherein the retaining ledge comprises the proximally directed surface.

25. The sub-assembly according to claim 16, further comprising a biasing member configured to bias the delivery member guard in the proximal direction relative to the housing from the distal position to the proximal position.

26. The sub-assembly according to claim 16, wherein the delivery member guard is configured to be manually moved from the distal position to the proximal position.

27. The sub-assembly according to claim 16, wherein one of the distally directed surface of the retainer and the proximally directed surface of the plunger rod is bevelled relative to the longitudinal axis.

28. A medicament delivery device comprising the sub-assembly according to claim 16, wherein the medicament delivery device comprises:
a power source at least partially arranged within the housing, wherein the power source is connected to the plunger rod, wherein the power source is configured to move the plunger rod in the proximal direction relative to the housing; and
a medicament container comprising a container body and a distal seal axially movable relative to the container body, wherein the medicament container is proximally positioned relative to the plunger rod within the housing,
wherein when the delivery member guard is in the proximal position, the plunger rod is configured to be moved by the power source to urge the distal seal of the medicament container in the proximal direction relative to the container body.

29. The medicament delivery device according to claim 28, wherein the power source is a spring.

30. The medicament delivery device according to claim 28, wherein the medicament delivery device comprises a delivery member assembly, wherein the medicament container comprises a pierceable proximal seal, wherein the delivery member assembly comprises a delivery member hub, a delivery member attached to the delivery member hub, and an attaching member attached to the proximal end of the housing, and wherein the delivery member hub is connected to the attaching member via a bayonet connection or a helical connection so that the delivery member hub can be manually moved towards the pierceable proximal seal relative to the housing.
